(19) United States
(12) Patent Application Publication
(10) Pub. No.: US 2025/0069697 A1
(43) Pub. Date: Feb. 27, 2025

(54) AN IMPROVED METHOD TO IDENTIFY NUCLEIC ACID SEQUENCES WITHIN A SET OF SEQUENCES OBTAINED BY A SEQUENCER AND A SYSTEM

(71) Applicant: INNOVACIÓN Y DESARROLLO DE ENERGÍA ALFA SUSTENTABLE, S.A. DE C.V., Nuevo León (MX)

(72) Inventors: Iván Alejandro DE LA PEÑA MIRELES, Monterrey (MX); José Luis ELIZONDO MURILLO, Monterrey (MX); Claudio GARIBAY ORIJEL, Metepec (MX); Javier ACEDO ZUÑIGA, La Paz (MX)

(73) Assignee: INNOVACIÓN Y DESARROLLO DE ENERGÍA ALFA SUSTENTABLE, S.A. DE C.V., Nuevo León (MX)

(21) Appl. No.: 18/724,910

(22) PCT Filed: Sep. 8, 2022
(86) PCT No.: PCT/MX2022/050074

§ 371 Date: Jun. 27, 2024

(30) Foreign Application Priority Data
Jan. 3, 2022 (MX) MX/A/2022/000217

(51) Int. Cl. G16B 30/00

(52) U.S. Cl. CPC G16B 30/00

(57) ABSTRACT

The present invention provides an improved method and system for identifying nucleic acid sequences within a set of sequences obtained by a sequencer, constructing database according to the identification of nucleic acid sequences, identifying different genus, species, sub-species, serotypes, variety of microorganism, virus, genes, or nucleic acid sequences of interest, for its use in the field of molecular biology applied to diagnosis, in hospitals, schools, industry or any place wherein this method and system is required to identify nucleic acid sequences, obtained by a sequencer. Specifically, the present invention provides an improved method and system that allow sequences to be differentiated from data obtained by nucleic acid sequencing.

### FIELD OF THE INVENTION

The present invention relates to an improved method and system to identify nucleic acid sequences present in a set of sequences, obtained by a sequencer, with the purpose of identifying different genus, species, subspecies, serotypes, varieties of organisms, viruses, genes or nucleic acid sequences of interest, for its use in the field of molecular biology, applied to diagnosis in hospitals, schools, industry or anywhere where identify nucleic acid sequences present in a set of sequences obtained by a sequencer is required. Specifically, this patent is related to an improved method and system that allow sequences to be differentiated from data obtained by nucleic acid sequencing

### BACKGROUND OF THE INVENTION

In the past years, nucleic acid sequencing has evolved from analyzing a sequence per reaction to millions of sequences per reaction. This type of sequencing is well-known as Next Generation Sequencing (NGS). NGS has reduced sequencing costs and has made the possibility to analyze samples that would not have been able to process previously due to its excessive costs.

The blood culture samples can be sequenced by NGS technology, making it especially useful to identify pathogen microorganism causing sepsis or their antibiotic resistance. This approximation can result in less aggressive, more adequate, and accurate therapies to patients in the treatment of sepsis, which could help to reduce the costs associated to the illness, which causes more than 270,000 deaths in the United States (Fay et al., 2020), and a bill of $44 US billon (Buchman et al., 2020).

Nevertheless, for this purpose it is required to use a system and a method capable of identifying sequences quickly and accurately, avoiding inaccurate results or incomplete information, which would result in an excessive delay to support therapies.

Some systems and methods that perform these types of tasks are based on similarities between two sequences, while others conduct a previous “mold” sequence for this purpose.

Some examples of systems and methods that are based on similarity between two sequences are the Needleman-Wunch (Needleman and Wunsch, 1970), the Smith-Waterman (Smith and Waterman, 1981), and other based on hide models are the Markov (Byung-Jun 2009), likewise the based on structure, such as the Fiser (Fiser, 2010). These systems and methods are generally used when two nucleic acid sequences or proteins are required to be compared with each other, regardless of their size. During the data processing step, statistical operations that use the sequences by themselves are included in the method, such as number of k-mer in each sequence, or similarity evaluation applying a matrix or arrays.

The main drawback about using one of these methods is that those can become inefficient when comparing two or more sequences, increasing the number of comparisons for aligning. Another drawback is that analysis does not consider the position of the sequences.

Examples of the systems and methods applied to analyze sequences are those which include a previous sequence or database as “mold” to identify new sequences, local alignments, and global alignments. (Polyanovsky et al., 2011), multiple sequences alignment (Chatzou et al., 2016), or manipulation of alignment (Benothman et al., 2008). Systems that utilize these methods consider the sequence position to make the alignment and can be applied to analyze more than two sequences. Nevertheless, those methods lose efficiency because the response time increases considerably. These types of systems and methods use devices such as computers wherein information is processed sequentially, which are limited by processing capacity that system and methods can provide (Chatzou et al., 2016).

Taking all of the above into consideration, the improved method and system from the present invention allow and are capable of identifying nucleic acid sequences within diverse types of sequenced samples by NGS. The improved method and system solve the analysis and processing problems of millions of sequences and their identification completing the task in the order of minutes.

### BRIEF DESCRIPTION OF THE INVENTION

The present invention relates to an improved method and system to identify nucleic acids sequences of a set of sequences obtained from a sequencer.

The main purpose of the invention is related to providing an improved method and system to identify specific nucleic acid sequences within a set of sequences obtained by any method of sequencing using NGS, making a metagenomic analysis.

Another purpose of the present invention relates to providing an improved method and system that allows processing data obtained by sequencing to identify specific nucleic acid sequences from any sequencer, in which the system could include one or more selected devices from the group consisting of computer (personal), lap-top, tablet, iPad, iPhone, mobile phone, smartphone, a server or other system able to process systematic operations.

Another purpose of the invention relates to providing an improved method and system that allows to build a database to identify specific nucleic acid sequences, selecting the highest similar sequences and evaluating iteratively until finding this similarity condition between sequences obtained by sequencing and the database sequences.

Another purpose of the invention relates to providing an improved method and system that allows to identify different genus, species, subspecies, serotypes, variety of organisms, virus, bacteria, genes, or any nucleic acid sequence of interest, found in a sequence sample obtained from any NGS sequencer.

### FIGURES DESCRIPTION

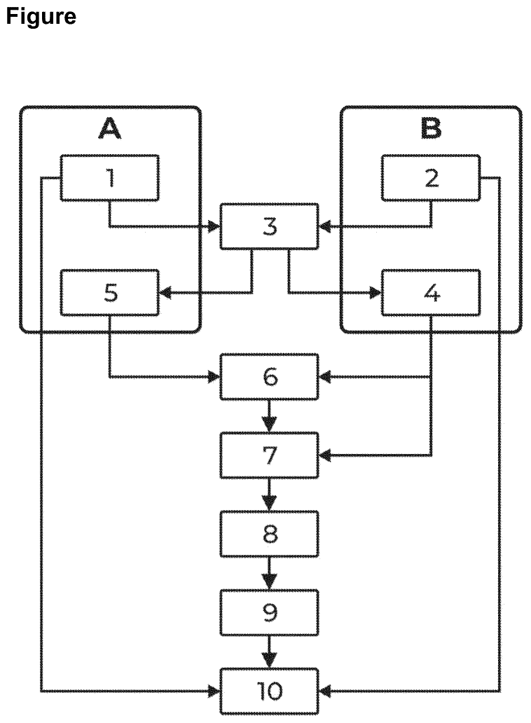

FIG. 1 Steps for identifying sequences obtained from a sequencer
A).—Corresponds to nucleic acids sequences obtained by means of a sequencer
B).—Corresponds to a specific nucleic acids sequence of reference obtained by means of a database, known by subject-matter experts;
1).—Data extraction obtained by a sequencer that comprises at least one or more specific sequences of DNA from a sample;
2).—Database building of specific sequences of reference obtained by means of existing databases known by subject-matter experts;
3).—Load of sequences obtained by means of a sequencer and the specific database of reference;
4).—Specific sequences of reference from the built database are converted in one or more Hash Tables;
5).—Obtaining a particular k-mer that represents each of the sequences obtained by means of a sequencer;
6).—k-mer from 5) are located in converted Hash Tables, excluding those sequences that don't have an associated position to the corresponding k-mer in the Hash table and selecting for their later analysis the sequences with one or more associated positions;
7).—The selected sequences are compared to specific sequences from the reference database, built according to positions obtained when Hash tables have been converted;
8).—The obtained sequences are analyzed in order by the position of the converted Hash tables and are joined to form a new consensus sequence;
9).—Determination of AGATA coefficient;
10).—Identification of the obtained sequences in 1) versus the database that was built in 2), applying the AGATA coefficient obtained in 9).

### DETAILED DESCRIPTION OF THE INVENTION

The present invention relates to an improved method and system for identifying nucleic acid sequences within a set of sequences, obtained by means of a sequencer with the purpose of identifying nucleic acid sequences, building databases based on the identification of nucleic acids sequences, and identify different genus, species, subspecies, serotypes, varieties of organisms, viruses, genes or nucleic acid sequences of interest, for use in the field of molecular biology applied to diagnosis in hospitals, schools, industry or anywhere where it is required to identify nucleic acid sequences present in a set of sequences. Specifically, it relates to an improved method and system that allows sequences to be differentiated from data obtained by nucleic acid sequencing.

### Definitions

Definitions are provided to better understand the object in the present invention.

The term “method” refers to an ordered set of stages and/or steps, and operations that allow the user to find the most similar sequences to each other, where the method may also include an “algorithm” in its stages and/or steps

The term “system” refers to the set of devices (components) and methods that allow the user to identify nucleic acid sequences, build databases based on the identification of nucleic acid sequences, and identify different genus, species, subspecies, serotypes, varieties of organisms, viruses, genes or nucleic acid sequences of interest, where the system uses one or more devices (components) selected from the group consisting of a personal computer, laptop, tablet, iPad, iPhone, mobile phone, smartphone, a server or other system that can process any set of operations or the combination of any of the above, with an application installed and/or to be installed.

The term “user” refers to the person who uses the system and method to identify nucleic acid sequences, build databases based on the identification of nucleic acid sequences, and identify different genus, species, subspecies, serotypes, varieties of organisms, viruses, genes, or nucleic acid sequences of interest according to the present invention.

The term “sequencer” refers to a device that is capable of chemically analyzing DNA and generating, as a result, a data file containing one or more DNA sequences.

The term “nucleotide” refers to the compounds of DNA, which can be adenine “A”, thymine “T”, guanine “G” and cytosine “C”.

The term “sequence” or “nucleic acid sequence” refers to an enumerated collection of nucleotides, which are part of DNA, regardless of their length, size, composition, or order.

The term “similarity” refers to the mathematical comparison between two or more sequences to determine how closely they resemble each other.

The term “metagenomic analysis” refers to the analysis performed on thousands of sequences by using a method, which allows certain patterns or characteristics defined by the user to be found.

The term “indexed data” refers to information that can be accessed by a number, key, or position in a list.

The term “database” refers to the set of indexed data belonging to the same context that contains records of names, numbers, passwords, dates and/or sequences, which are stored for its later access.

The symbol [symbol] refers to the set of all sequences.

The term “sequence length” refers to the number of nucleotides in the sequence.

The term “sequence contained in sequence” means that every nucleotide in the first sequence is found in the second sequence in the same order. Mathematically symbolized as, the sequence S is contained in the sequence T:

S⊂T

The term “k-mer” of a sequence A refers to a sequence contained in sequence A whose length is exactly k.

The term “Total k-mers” refers to the set consisting of all possible k-mers with the symbols {A, T, G, C}. Mathematically, is symbolized as:

Σ^{k}

The term “initial k-mer” of a sequence refers to the k-mer of the sequence with which the sequence begins whose length is exactly k.

The term “intersection of two sequences” refers to the matching sequence that is contained in both sequences. Mathematically it can be symbolized as:

A=S∩T Where: A⊂S and A⊂T

The term “end intersection of two sequences” refers to the intersection of two sequences from the left or right side of the sequences.

The term “concatenation” of two sequences refers to the operation of adding the symbols of one sequence to another. This concatenation differs whether it is from the left or from the right.

The term “end-overlapping concatenation of two sequences” refers to the sequence resulting from concatenating two sequences by overlapping the intersection of the sequences from some ending, either left or right. Mathematically written as:

S∪T=S′·A·T′

Where A is the overlapping part of the intersection of the rightmost sequences from S to T. S∪T≠T∪S, since T∪S involves the intersection of T with S from the right.

The term “function” refers to a relationship between elements of two sets that fulfills a certain correspondence rule, so that each element of the first set corresponds to a single element of the second.

The term “bijective function” refers to a function in which, if two elements of the first set are assigned the same element of the second, it concludes that the elements of them are equal and that for each element in the second set there is an element in the first to which it is related by the function.

The term “finitely countable” refers to the fact that there is a bijective function between the elements of a set and a finite subset of natural numbers.

For practical purposes, the sets of “Total k-mers” (Σ^{k}) are finitely countable with the subset {1, 2, . . . , 4^{k}} of natural numbers.

The term “Hash Table” refers to a data structure in which a certain position or access key allows returning values or specific information.

The term “Hash Table Representation of a Sequence” refers to a Hash Table containing all possible k-mers as access keys and the positions of a given sequence as values.

The term “Search by initial k-mer” refers to the access of the Hash Table Representation of a sequence, through the bijective function that induces its finite numbering of the Total k-mers.

The term “genus” refers to a classification of organisms widely known in the field of taxonomy.

The term “species” refers to a classification of organisms widely known in the field of taxonomy.

The term “serotype” refers to a classification of organisms widely known in the field of taxonomy. To determine a serotype, tests are usually done to detect certain proteins in the cell membrane or cell wall.

The term “variety” refers to a classification of organisms widely known in the field of taxonomy.

The term “sample” refers to any substance of any chemical composition, whether liquid, solid or gaseous, that contains organisms or DNA that can be subjected to analysis to determine the DNA sequences present in it.

The term “biological sample” refers to a sample obtained specifically from a tissue, organ, bone, blood, or fluid from a person.

The term “nucleic acid extraction” refers to a chemical procedure where a sample is mixed with chemicals to extract DNA.

The terms “gene” or “genes” refer to biological molecules composed of nitrogenous compounds or nitrogenous bases known in the state of the art as Adenine, Guanine, Cytosine and Thymine. Genes are the molecules that transmit information in a cell for the biological synthesis of RNA and later, if applicable, proteins, or enzymes.

The term “virus” refers to a set of proteins, lipids and genetic material that is structured and that when it comes into contact with a related cell, it can be infected and used to multiply.

Next, the theory and theorems that serve to define the improved method that will be used in the system of the present invention are described, as shown below:

### I. Sequence Theory

Let the alphabet Σ={A, T, G, C} for DNA sequences, where each sequence can be written as a concatenation of characters in the alphabet Σ.

Given a sequence A, we define the length of A as: |A| the number of letters or symbols that concatenated form A.

Let Σ^{α} as the set of all sequences of length α (base pairs). Thus, the set of all sequences [symbol] can be written as:

$$= {⋃}_{α = 0}^{∞} {Σ}^{α}$$

Let S∈

[symbol]

Given k a natural number, we define the initial k-mer (Ks) of a sequence S as the sequence of length k such that S=Ks·S1, where S1 is the sequence that completes S and ·s the concatenation operation of characters.

Given a natural number n. We define the “symbol n in S” S[n] as the character of S at position number n.

Let S, T∈

[symbol]

We define that a sequence S is contained in a sequence T (S⊂T) if there exist sequences A, B∈ [symbol] such that T=A·S·B, where · is the concatenation operation.

We define an intersection of two sequences to be a sequence A such that A⊂S and A⊂T.

We define the end intersection between S and T as a sequence A such that A is an intersection of S with T and there exist S1, S2, T1, T2ϵ [symbol] such that:

(Right end) S=S2·A and T=A·T1

or

(Left end) S=A·S1 and T=T2·A

We define the end-overlapping concatenation operation between two sequences as the result of obtaining the end-intersection of the two sequences and resulting in the concatenation:

S⋄T=S′·A·T′

For practical purposes it can be represented as S∪T, but denoting the difference of the non-commutativity between the sets.

Where A is the corresponding end intersection and S′, T′ are the partial sequences that define the end intersection itself.

### II. Theorem: (Base by End-Overlapping Concatenation)

Let Sϵ [symbol] be an arbitrary sequence. For every k natural number, there is a sequence Sₖ of elements in Σ^{k} such that S is an overlapping concatenation of the ends of all the elements of Sₖ in order.

### Demonstration:

Let express S=a₁a₂ . . . aₙ as a string of concatenated characters.

Let Sᵢ=aᵢaᵢ₊₁ . . . aᵢ₊ₖ₋₁, ∀i=1, . . . , n−k+1

Then, we have that:

$$\vert {S}_{i} \vert = ( k - 1 ) + 1 = k , ∀ i = 1 , … , n - k + 1$$

Therefore

$${S}_{i} ∈ {Σ}^{k} , ∀ i = 1 , … , n - k + 1$$

And it is verified inductively that:

$$( {S}_{1} ⋄ {S}_{2} ) … ⋄ {S}_{n - k + 1} = {a}_{1} {a}_{2} … {a}_{n} = S$$

Let 1≤i≤n−k+1

For i=2

$$( {S}_{1} ⋄ {S}_{2} ) = ( {a}_{1} {a}_{2} … {a}_{k} ) ⋄ ( {a}_{2} {a}_{3} … {a}_{k + 1} ) = ( {a}_{1} {a}_{2} … {a}_{k + 1} )$$

So a₂ . . . aₖ is an end intersection between S₁ and S₂

Suppose it is valid for a fixed i ((S₁ ⋄ S₂) . . . ⋄ Sᵢ)=(a₁a₂ . . . aₖ₊₍ᵢ₋₁₎), as an example, it is described the demonstration for i+1

$$( {S}_{1} ⋄ {S}_{2} ) … ⋄ {S}_{i + 1} = ( ( {S}_{1} ⋄ {S}_{2} ) … ⋄ {S}_{i} ) ⋄ {S}_{i + 1} = ( {a}_{1} {a}_{2} … {a}_{k + ( i - 1 )} ) ⋄ {S}_{i + 1} = ( {a}_{1} {a}_{2} … {a}_{k + ( i - 1 )} ) ⋄ ( {a}_{i + 1} {a}_{i + 2} … {a}_{k + i} ) = ( {a}_{1} {a}_{2} … {a}_{k + i} )$$

With aᵢ₊₁aᵢ₊₂ . . . aₖ₊₍ᵢ₋₁₎ an end intersection between the sequence (a₁a₂ . . . aₖ₊₍ᵢ₋₁₎) and (aᵢ₊₁aᵢ₊₂ . . . aₖ₊ᵢ)
Q.E.D.

### III. Theorem

There is a bijection between Σ^{k} and {0, 1, 2, . . . , 4^{k}−1} that induces an ordering between the elements of Σ^{k}

Demonstration:

Let S=a₁a₂ . . . aₖ∈Σ^{k}

Let a function ƒ which assigns numerical values for the letters of the alphabet that form the sequences.

ƒ: {A,T,G,C}→{0,1,2,3}|ƒ(A)=0,ƒ(T)=1,ƒ(G)=2,ƒ(C)=3

Note that by definition ƒ is bijective

Define g: Σ^{k}→{0, 1, . . . 4^{k}−1}, such that

$$g ( S ) = g ( {a}_{1} {a}_{2} … {a}_{k} ) = {\sum }_{i = 1}^{k} {4}^{i - 1} f ( {a}_{i} )$$

We show that g is bijective too.

### If

$$g ( A ) = g ( B ) \\ {\sum }_{i = 1}^{k} {4}^{i - 1} f ( {a}_{i} ) = {\sum }_{i = 1}^{k} {4}^{i - 1} f ( {b}_{i} ) \\ {4}^{k - 1} f ( {a}_{k} ) + … + {4}^{0} f ( {a}_{1} ) = {4}^{k - 1} f ( {b}_{k} ) + … + {4}^{0} f ( {b}_{1} )$$

Taking integer divisions by 4^{k-1} on both sides, we have

$${4}^{k - 1} f ( {a}_{k} ) / {4}^{k - 1} = {4}^{k - 1} f ( {b}_{k} ) / {4}^{k - 1}$$

Because every 4ⁱ⁻¹ ƒ(aᵢ)<4^{k-1}, for all i∈{1, . . . , k−1},

with ƒ(aᵢ)<4

Then:

ƒ(aₖ)=ƒ(bₖ)

It follows

$${4}^{k - 1} f ( {a}_{k} ) = {4}^{k - 1} f ( {b}_{k} ) \\ {4}^{k - 2} f ( {a}_{k - 1} ) + … + {4}^{0} f ( {a}_{1} ) = {4}^{k - 2} f ( {b}_{k - 1} ) + … + {4}^{0} f ( {b}_{1} )$$

Thus

Now, integer divisions between 4^{k-2} are taken, so on. Since we have a finite number of values, we get that

ƒ(a₁)=ƒ(bᵢ),∀i∈{1, . . . ,k}

Then

A=B

Therefore g is injective

Now, let x∈{0, 1, 2, . . . , 4^{k-1}}

For 4^{k-1}, by the division theorem, there exists q₁, r₁, 0≤r₁<4^{k-1}, such that:

$$x = {4}^{k - 1} {q}_{1} + {r}_{1}$$

Where, if x<4^{k} then 0≤q₁<4

Now for r₁ y 4^{k-2}, there exist q₂, r₂, 0≤r₂<4^{k-2}, such that:

$${r}_{1} = {4}^{k - 2} {q}_{2} + {r}_{2}$$

Where, if r₁<4^{k-1} then 0≤q₂<4

So

$$x = {4}^{k - 1} {q}_{1} + {4}^{k - 2} {q}_{2} + {r}_{2}$$

Successively, we get that

$$x = {4}^{k - 1} {q}_{1} + {4}^{k - 2} {q}_{2} + … + {4}^{0} {q}_{k} \\ 0 \leq {q}_{i} < 4 , ∀ i ∈ \{ 1 , … , k \}$$

Where

As the function ƒ, is bijective by definition for each element qᵢ∈{0, 1, 2, 3}, there exists aᵢ∈{A, T, G, C}, such that qᵢ=ƒ(aᵢ), so we can express

$$x = {4}^{k - 1} f ( {a}_{1} ) + {4}^{k - 2} f ( {a}_{2} ) + … + {4}^{0} f ( {a}_{k} ) = {\sum }_{i = 1}^{k} {4}^{i - 1} f ( {a}_{k + 1 - i} ) = g ( A )$$

Where A=aₖaₖ₋₁ . . . a₂a₁∈Σ^{k}

Therefore g is surjective and therefore is bijective.

Then g induces a good order of the numbers in {0, 1, 2, . . . , 4^{k}−1}, given by:

Let S, T∈Σ^{k}, we say that S<T if and only if g(S)<g(T)
Q.E.D

### Corollary: (Hash Representation)

Given k a natural number. Every sequence can be represented as an enumerated collection of indexes on {1, 2 . . . , 4^{k}}. Furthermore, every sequence can be written as a list of indexes of exactly 4^{k} options

Let Sϵ

[symbol]

Applying what we have seen in the theorems:

There exist {Sᵢ} enumerated collection of elements in Σ^{k}, such that S is a “end-overlapping concatenation” of elements in {Sᵢ}.

Using the bijective function g from the previous theorem, then the enumerated collection {g(Sᵢ)} is an enumerated collection of elements in {0, 1, 2 . . . , 4^{k}−1}, which identifies the sequence S

Without loss of generality, we can reindex the elements of {0, 1, 2 . . . , 4^{k}−1} in {1, . . . , 4^{k}}, which is the enumerated collection {g(Sᵢ)}, by having possible repeated values, it can be arranged on a table of exactly 4^{k} different options, where each option x∈{1, . . . , 4^{k}} lists the elements with j-index that form the sequence, such that x=g(Sⱼ).

### IV. Theorem: (Search by Anchor)

Given two sequences S,Tϵ [symbol] and k a natural number, such that |S|>|T|, there exists an index λ such that the initial k-mer of T can be found in all possible position of S.

### Demonstration:

Let X the initial k-mer of T, and by the above corollary let L_{S} the representation of the positions of S in all its different k-mers that compose it.

Then using the same function g which induces its representation of L_{S}.

Let λ=g(X), then the index λ found in representation L_{S} corresponds to the list of all possible positions of X in S.

Let R be the reference sequence to be compared, by the theorems that we have reviewed there is a function ƒ that is bijective and maps the reference sequence in a representation of indexes or Hash table H_{R}

That is, given a fixed number k, all reference sequences can be converted into ordered hash tables in which the exact positions at which each of the possible k-mers for DNA sequences are located are listed (The conversion results in 4^{k} elements in the Hash Table).

A filtered subset of the set of test sequences is obtained for each alignment.

Let R the reference sequence and let:

$${S}_{T} = \{ X ∈ \vert X is a test sequence \}$$

The set of all test sequences for an alignment.

Given a fixed but arbitrary natural number k, a collection of disjoint k-mers of the reference sequence to be aligned is constructed

$${P}_{k} = \{ X ⊂ Y \vert X is a k - mer of R with Y ∈ {S}_{T} \}$$

Fulfilling that given

Xᵢ,Xⱼ∈Pₖ si Xᵢ≠Xⱼ→Xᵢ∩Xⱼ=∅

### (Completely Different Sequences)

If α is the reference sequence, the subset of test sequences is defined as:

$${S}_{T} ( α ) = \{ X ∈ {S}_{T} \vert X contains some k - mer of {P}_{k} \}$$

Through this set, we can work with the initial k-mers of each sequence in S_{T}(α), to locate them using the representations L_{R} of each reference sequence R, to select the corresponding indexes of the positions of each sequence according to what is seen in the theorems.

The Sorensen-Dice coefficient is calculated (seen from the perspective of intersection and union of sequences) between two DNA sequences of the same size (Forcing the measure by subsequences on the sequences without gaps), reducing it to an operation of the number of pairs of bases shared by the sequences, divided by the total number of base pairs in the sequences.

$$D ( A , B ) = \frac{2 \vert A ⋂ B \vert}{\vert A \vert + \vert B \vert} ( Sorensen - Dice coefficient )$$

In this case, since it is handled that the measures of the sequences are equal |A|=|B|

$$D ( A , B ) = \frac{2 \vert A ⋂ B \vert}{2 \vert A \vert} = \frac{\vert A ⋂ B \vert}{\vert A \vert}$$

This coefficient is calculated for each subsequence of the reference sequences identified by the initial k-mer (with the index k defined in the construction stage) of exact size using the hash table representation. According to an evaluation system, the sequences with the highest value in their similarity evaluation are selected and saved.

The evaluation of the “AGATA Coefficient” of a complete reference DNA sequence against a set of test sequences is generalized as the global Sorensen-Dice coefficient induced by the consensus alignment (“overlapping concatenation” of all test sequences selected by the evaluation system) in the corresponding positions according to the hash table.

Let α be the reference sequence, let S_{T}(α) the set of test sequences for the multiple alignment of α and AG the subset of S_{T}(α) of the sequences selected by the evaluation system. Then:

The consensus sequence C can be represented as follows, since AG is finite of cardinality say n, we can number the set and substitute the indices at the union (overlapping concatenation)

$$C = {⋃}_{{S}_{s} ∈ AG} {S}_{s} = {⋃}_{k = 1}^{n} {S}_{k}$$

Thus, the AGATA Coefficient (AC), can be defined as a similarity index of a set of multiple sequences, applied to a specific sequence present in a database, where:

$$CA ( α , {S}_{T} ( α ) ) = D ( α , C ) = \frac{2 \vert α ⋂ C \vert}{\vert α \vert + \vert C \vert}$$

Whereby construction |α|=|C|

$$CA ( α , {S}_{T} ( α ) ) = D ( α , C ) = \frac{2 \vert α ⋂ C \vert}{2 \vert α \vert} = \frac{\vert α ⋂ C \vert}{\vert α \vert} \\ CA ( α , {S}_{T} ( α ) ) + D ( α , C ) = \frac{2 \vert α ⋂ C \vert}{2 \vert α \vert} = \frac{\vert {⋃}_{{S}_{s} ∈ AG} α ⋂ {S}_{s} \vert}{\vert α \vert}$$

By further detailing the expression, by the inclusion-exclusion principle, the AGATA Coefficient is defined as:

$$CA ( α , {S}_{T} ( α ) ) = {\sum }_{k = 1}^{n} {( - 1 )}^{k + 1} \frac{\vert {⋂}_{i ∈ I ⊂ \{ 1 , … , n \} : \vert I \vert = k} {S}_{i} \vert}{\vert α \vert}$$

In a first embodiment of the present invention the improved method to identify nucleic acids sequences within a set of sequences, obtained by means of a sequencer comprises the following steps:
Step 1).—Extraction; data is extracted from a sequencer that comprises at least one or more specific sequences of DNA of a sample, where the obtained data are one or more nucleic acids sequences;
Step 2).—Database of specific sequences building; specific nucleic acids sequence of reference is obtained from known databases, which this specific database of sequences of reference is identified by the name of the gene or DNA fragment;
Step 3).—Sequences loading; obtained sequences are loaded in step 1 along with database in step 2;
Step 4).—Hash table conversion; specific sequences of reference from the database that was built in step 2 is converted in one or more hash tables; where the specific sequence of reference of databases are converted in an exact position lists which each possible k-mer is located and where the table size has a number of elements equal to 4^{k}; the k value is a positive integer number fixed by the user, the k value is preferred between 1 and 30, more preferred between 7 and 15, and more preferred between 9 and 12;
Step 5).—Obtaining of representative k-mers from the obtained sequences in step 1, an individual k-mer is obtained which represents each sequence obtained in step 1, wherein the k-mer size could be equal or/and different than selected k-mer for each Hash tables converted in step 4, wherein the k value is preferred between 1 and 30, more preferred between 7 and 15, and more preferred between 9 and 12;
Step 6).—Selection and exclusion, k-mers obtained in step 5 are located in Hash tables converted in step 4, excluding sequences not having an associated position to a correspondent k-mer, and selecting for its later analysis the sequences having one or more positions associated to the Hash table obtained in step 4;
Step 7).—Evaluation, selected sequences in step 6 are compared to the specific sequences of reference of the database that was built in step 2, according to the positions obtained from converting Hash tables in step 4, wherein the comparison of one of two sequences must accomplish an evaluation criteria defined by the user, where the criteria of evaluation defined by the user is equal to or greater than 90% of similarity;
Step 8).—Determination of consensus of sequences, sequences obtained in step 7 are analyzed in order by the position of Hash tables converted in step 4, applying the following expression:

S∪T=S′·A·T′

Where A is the overlapping part of the intersection of the rightmost sequences from S to T, to generate the consensus sequence;
Step 9).—Determination of AGATA coefficient, sequences obtained in step 7 are utilized to determine AGATA coefficient, wherein AGATA coefficient is a decimal number between 0 and 1, wherein 0 is the absence of similarity (0%) and 1 is the full similarity, where AGTATA coefficient is defined by the following expression:

$$CA ( α , {S}_{T} ( α ) ) = {\sum }_{k = 1}^{n} {( - 1 )}^{k + 1} \frac{\vert {⋂}_{i ∈ I ⊂ \{ 1 , … , n \} : \vert I \vert = k} {S}_{i} \vert}{\vert α \vert}$$

where α is a specific sequence of reference of the databases that were built in step 2, S_{T}(α) are the sequences selected in step 7 and C is the consensus sequence obtained in step 8 defined by

$$C = {⋃}_{k = 1}^{n} {S}_{k}$$

the above allows to obtain the AGATA coefficient for each of the specific sequences of reference of the databases that were built in step 2;
Step 10).—Identification, AGATA coefficients obtained in step 9 allows to identify nucleic acid sequences obtained in step 1 with higher similarity to the specific sequences of reference of the databases that were built in step 2.

In a second embodiment of the present invention, the method can identify bacteria genes, wherein the database that was built in step 2 comprises nucleic acids sequences from bacteria genes.

In a third embodiment of the present invention, the method can identify yeasts genes, wherein the database that was built in step 2 comprises nucleic acids sequences from yeasts genes.

In a fourth embodiment of the present invention, the method can identify virus genes, wherein the database that was built in step 2 comprises nucleic acids from virus genes.

In a fifth embodiment of the present invention, the method can identify fungus genes, wherein the database that was built in step 2 comprises nucleic acids from fungus genes.

In a sixth embodiment of the present invention, the method is capable of identifying plant genes, wherein the database that was built in step 2 comprises nucleic acid sequences from plant genes.

In a seventh embodiment of the present invention, the method can identify animal genes, wherein the database that was built in step 2 comprises nucleic acid sequences from animal genes.

In an eighth embodiment of the present invention, the method can identify human genes, wherein the database that was built in step 2 comprises nucleic acid sequences from human genes.

In a ninth embodiment of the present invention, the method can identify microorganism that cause sepsis, wherein the database that was built in step 2 comprises nucleic acid sequences from microorganism that cause sepsis.

In a tenth embodiment of the present invention, the method can identify resistance genes to antibiotics, wherein the database that was built in step 2 comprises nucleic acid sequences from resistance genes to antibiotics.

In an eleventh embodiment of the present invention, is referred to the system that using and analyzing metagenomic data obtained by means of a sequencer, is capable of identifying specific nucleic acid sequences, allowing the user to modify the search options, generating and modifying databases which allows finding different nucleic acid sequences, wherein the improved system comprises the following elements:
i. The improved method described in the present invention, compatible with system devices;
ii. A hard disk where the files are saved and stored related with the improved method and the database that the system utilizes;
iii. RAM with processability for loading and accessing databases; processing depends on database size, with a minimum of 2 GB;
iv. Processing unit that comprises at least 2 physical cores for a continuous operation of the improved method;

In a twelfth embodiment of the present invention, is referred to an improved system that comprises the following elements:
I. One or more servers and/or processing units that have the database loaded, communicated with each other through a digital network;
II. A central server and/or a central processing unit that have the database loaded, and communicate to computers, lap-top, tablets, iPad, iPhone, mobile phone, smartphones, or other server or system which could process a set of systematic operations through an installed application or app, to load, update, refresh, and delete data, to respond to informatic requests by the user, deploy, and/or share information and requests.
III. One or more devices selected from the group that comprises computer, lap-top, tablet, iPad, iPhone, mobile phone, smartphone, a server or other system able to process a set of systematic operations through and installed application or app.
IV. A digital informatic network that allows communication and link-up with servers, central server, or devices.

In a thirteen embodiment of the present invention, the improved system comprises the following components:
A processor unit or processer that has a computer-readable medium that causes the processor to perform a method to identify nucleic acid sequences of the present invention.

### EXAMPLES

The following examples are intended to clarify the novelty and inventiveness of the present invention. It must be understood that the following examples do not constitute a limit of the scope of the present invention. From invention description as well as from the following examples, a person having an ordinary skill in the field of the invention can make modifications, that anyway remains within the framework protected of the present invention.

### Example 1. Microorganism Identification that Causes Sepsis Applying Microorganism Isolated from Blood Cultures

The following example is intended to show that the improved method and system of the present invention identify and/or detect sepsis-causing microorganisms that have been previously isolated from human blood cultures.

Blood samples from patients infected and with sepsis were collected in blood culture collection bottles. Samples were placed in the equipment BD BACTEC™ FX40 (Becton Dickinson Company), for determining if the blood was contaminated with microorganism.

When the culture turned positive in bacteria presence, an aliquot was seeding in Trypticasein Soy Agar Plates.

When the isolated colonies were obtained, the genus and/or the specie was determined applying two strategies:
a) Biochemical testing

Biochemical testing applied were the widely known in the state-of-art for microbiological identifying, giving examples: Catalase, Oxidase, Aminopeptidase, Urease, Indole, Nitrite Reduction, Methyl Red, Voges-Proskauer, TSI, etc.
b) DNA extraction, sequencing, and identification utilizing the method (FIG. 1) and the system proposed in the present invention.

DNA extraction was done using the Wizard® Genomic DNA Purification Kit (Promega Inc.), following the manufacturer instructions. DNA sequencing was done through the Miniseq Sequencing System (Illumina Inc.), following the manufacturer instructions.

In order to use the improved method and system of the present invention, a database was loaded with the nucleic acid sequences obtained in public databases known by the subject-matters experts (pubmed, kegg, among others, etc.) described in Table 1.

TABLE 1
Database to identify microorganism that causes sepsis

| Gene | Genus | Species |
|---|---|---|
| 16SrDNA | 28 | 1,466 |
| rpoB | 28 | 1,007 |
| recA | 28 | 821 |
| GyrB | 28 | 917 |

The system of the present invention uses one or more devices selected from de group set comprising computer, lap-top, tablet, iPad, iPhone, smartphone, and a server

Overall, eighty samples were analyzed for identification using both biochemical testing and the proposed method in the present invention. In all systems that were utilized, equal results were obtained. As an example, in Table 2 the obtained results by both biochemical testing and the improved method of the present invention using a tablet are shown and it can be appreciated that both methods resulted in the same identification of genus, nevertheless, the improved method could identify species efficiently since some bacteria were identified just up to genus with biochemical testing, such as the samples 6, 20, 21, 25, 26, 42, 43, 50, 51, 53, 54, 59, 62, 65, 68, 70 y 79.

TABLE 2
Microorganism Identification through both biochemical testing
and the improved method of the present invention.

| | | | Proposed method of the | |
|---|---|---|---|---|
| | Biochemical testing | | present invention | |
| Sample | Genus | Specie | Genus | Specie |
| 1 | Staphylococcus | aureus | Staphylococcus | Aureus |
| 2 | Staphylococcus | epidermidis | Staphylococcus | epidermidis |
| 3 | Staphylococcus | epidermidis | Staphylococcus | epidermidis |
| 4 | Enterococcus | faecalis | Enterococcus | faecalis |
| 5 | Staphylococcus | aureus | Staphylococcus | aureus |
| 6 | Staphylococcus | Sp | Staphylococcus | aureus |
| 7 | Enterococcus | faecalis | Enterococcus | faecalis |
| 8 | Enterococcus | faecalis | Enterococcus | faecalis |
| 9 | Staphylococcus | aureus | Staphylococcus | aureus |
| 10 | Escherichia | coli | Escherichia | coli |
| 11 | Serratia | marcescens | Serratia | marcescens |
| 12 | Serratia | marcescens | Serratia | marcescens |
| 13 | Staphylococcus | aureus | Staphylococcus | aureus |
| 14 | Staphylococcus | aureus | Staphylococcus | aureus |
| 15 | Enterococcus | faecalis | Enterococcus | faecalis |
| 16 | Staphylococcus | aureus | Staphylococcus | aureus |
| 17 | Klebsiella | pneumoniae | Klebsiella | pneumoniae |
| 18 | Escherichia | coli | Escherichia | coli |
| 19 | Klebsiella | pneumoniae | Klebsiella | pneumoniae |
| 20 | Staphylococcus | Sp | Staphylococcus | epidermidis |
| 21 | Enterococcus | sp. | Enterococcus | faecium |
| 22 | Klebsiella | pneumoniae | Klebsiella | pneumoniae |
| 23 | Staphylococcus | aureus | Staphylococcus | aureus |
| 24 | Staphylococcus | aureus | Staphylococcus | aureus |
| 25 | Enterococcus | sp. | Enterococcus | faecalis |
| 26 | Enterococcus | sp. | Enterococcus | faecalis |
| 27 | Enterococcus | faecalis | Enterococcus | faecalis |
| 28 | Staphylococcus | aureus | Staphylococcus | aureus |
| 29 | Enterococcus | faecalis | Enterococcus | faecalis |
| 30 | Enterococcus | faecalis | Enterococcus | faecalis |
| 31 | Enterococcus | faecalis | Enterococcus | faecalis |
| 32 | Enterococcus | faecalis | Enterococcus | faecalis |
| 33 | Staphylococcus | aureus | Staphylococcus | aureus |
| 34 | Klebsiella | pneumoniae | Klebsiella | pneumoniae |
| 35 | Klebsiella | pneumoniae | Klebsiella | pneumoniae |
| 36 | Enterococcus | faecalis | Enterococcus | faecalis |
| 37 | Pseudomonas | aeruginosa | Pseudomonas | aeruginosa |
| 38 | Serratia | marcescens | Serratia | marcescens |
| 39 | Pseudomonas | aeruginosa | Pseudomonas | aeruginosa |
| 40 | Escherichia | coli | Escherichia | coli |
| 41 | Escherichia | coli | Escherichia | coli |
| 42 | Staphylococcus | sp. | Staphylococcus | aureus |
| 43 | Staphylococcus | sp. | Staphylococcus | aureus |
| 44 | Pseudomonas | aeruginosa | Pseudomonas | aeruginosa |
| 45 | Pseudomonas | aeruginosa | Pseudomonas | aeruginosa |
| 46 | Escherichia | coli | Escherichia | coli |
| 47 | Klebsiella | pneumoniae | Klebsiella | pneumoniae |
| 48 | Pseudomonas | aeruginosa | Pseudomonas | aeruginosa |
| 49 | Escherichia | coli | Escherichia | coli |
| 50 | Enterococcus | sp. | Enterococcus | faecalis |
| 51 | Staphylococcus | sp. | Staphylococcus | epidermidis |
| 52 | Escherichia | coli | Escherichia | coli |
| 53 | Staphylococcus | sp. | Staphylococcus | aureus |
| 54 | Staphylococcus | sp. | Staphylococcus | aureus |
| 55 | Klebsiella | pneumoniae | Klebsiella | pneumoniae |
| 56 | Klebsiella | pneumoniae | Klebsiella | pneumoniae |
| 57 | Serratia | marcescens | Serratia | marcescens |
| 58 | Serratia | marcescens | Serratia | marcescens |
| 59 | Staphylococcus | sp. | Staphylococcus | hominis |
| 60 | Staphylococcus | aureus | Staphylococcus | aureus |
| 61 | Klebsiella | pneumoniae | Klebsiella | pneumoniae |
| 62 | Staphylococcus | sp. | Staphylococcus | epidermidis |
| 63 | Klebsiella | pneumoniae | Klebsiella | pneumoniae |
| 64 | Klebsiella | pneumoniae | Klebsiella | pneumoniae |
| 65 | Pseudomonas | sp. | Pseudomonas | aeruginosa |
| 66 | Staphylococcus | aureus | Staphylococcus | aureus |
| 67 | Klebsiella | pneumoniae | Klebsiella | pneumoniae |
| 68 | Staphylococcus | sp. | Staphylococcus | epidermidis |
| 69 | Klebsiella | pneumoniae | Klebsiella | pneumoniae |
| 70 | Acinetobacter | sp. | Acinetobacter | baumannii |
| 71 | Klebsiella | pneumoniae | Klebsiella | pneumoniae |
| 72 | Klebsiella | pneumoniae | Klebsiella | pneumoniae |
| 73 | Klebsiella | pneumoniae | Klebsiella | pneumoniae |
| 74 | Proteus | mirabilis | Proteus | mirabilis |
| 75 | Klebsiella | pneumoniae | Klebsiella | pneumoniae |
| 76 | Acinetobacter | calcoaceticus | Acinetobacter | baumannii |
| 77 | Klebsiella | pneumoniae | Klebsiella | pneumoniae |
| 78 | Klebsiella | pneumoniae | Klebsiella | pneumoniae |
| 79 | Staphylococcus | sp. | Staphylococcus | epidermidis |
| 80 | Serratia | marcescens | Serratia | marcescens |

This example showed that bacteria present in blood previously isolated were identified effectively applying the improved system and method of the present invention in comparison with biochemical testing, in which not all the species were identified.

### Example 2. Identification of Microorganisms in Blood Samples without Bacteria Isolation and Identification of Genes that Causes Antibiotic Resistance

The following example is intended to demonstrate that the improved method and system of the present invention identify and/or detect sepsis-causing microorganism and their genes that causes antibiotic resistance without the need of a previous bacteria isolation.

Blood samples from human patients were collected in blood culture collection bottles. Blood culture bottles were placed in the equipment BD BACTEC™ FX40 (Becton Dickinson Company) for determining microorganism presence in blood.

When the culture turned positive to bacteria presence, 3 mL were used for nucleic acid extraction applying the Wizard® Genomic DNA Purification Kit (Promega Inc.), following the manufacturer instructions. DNA sequencing was done through the Miniseq Sequencing System (Illumina Inc.), following the manufacturer instructions.

For this example, only results obtained when the system was applied by a laptop as selected device were shown, nevertheless, it is important to mention that applying every device listed and described above in the present invention, resulted in the same output. The improved method and system of the present invention utilized a database of genes that allows the identification of microorganisms and their antibiotic resistance genes, described in Table 3. The total of genes utilized for this database were 4,594.

TABLE 3
Database to identify sepsis-causing microorganism
and their antibiotic resistance genes

| Gene | Total of sequences |
|---|---|
| Genes to identify microorganism | 4,211 |
| Genes to identify antibiotic resistance | 383 |

In order to prove that the improved method and system proposed in the present invention works to predict resistance or susceptibility to antibiotics, some microorganisms were isolated in Trypticasein Soy Agar. Subsequently, the isolated were seed in antibiotic mediums for which the microorganism showed susceptibility and resistance.

Five blood samples that showed bacteria growth in the blood culture were analyzed utilizing the improved method and system of the present invention. The identification of genus and specie utilizing both the biochemical testing and the method of the present invention were the same. The results are described below:
Sample 1: The identified microorganism utilizing the improved method proposed in the present invention was Klebsiella pneumoniae. The identification time of genus and specie, and its antibiotic resistance genes was 10 minutes. The results showed that this genus and specie identified utilizing the method and system have a resistance to these antibiotics: Cephalosporins, Cephamycin, Fluoroquinolones, Monobactam, Penam, Penem and Tetracyclines.
Sample 2: The identified microorganism utilizing the improved method proposed in the present invention was Staphylococcus aureus. The identification time of genus, specie and its antibiotic resistance genes was 12 minutes. The antibiotics for which this microorganism presents resistance based on the results of the improved system in the present invention are Macrolides, streptogramins, Tetracyclines, Fluoroquinolones and Penam.
Sample 3: The identified microorganism utilizing the improved method proposed in the present invention was Enterococcus faecalis. The identification time of genus, specie and its antibiotic resistance genes was 7 minutes. The antibiotics for which this microorganism presents resistance based on the results of the improved system in the present invention are Diaminopyrimidine, Lincosamides, Pleuromutilin, Streptogramin, Tetracyclines.
Sample 4: The identified microorganism utilizing the improved method proposed in the present invention was Staphylococcus epidermidis. The identification time of genus, species and its antibiotic resistance genes was 15 minutes. The antibiotics for which this microorganism presents resistance based on the results of the improved system in the present invention are Carbapenem, Cephalosporins, Monobactams, Penam, Fluoroquinolones, Macrolides, Streptogramins.
Sample 5: The identified microorganism utilizing the improved method proposed in the present invention was Escherichia coli. The identification time of genus, specie and its antibiotic resistance genes was 5 minutes. The antibiotics for which this microorganism presents resistance based on the results of the improved system in the present invention are Cephalosporins, Fluoroquinolones, Lincosamides, Macrolides, Monobactam, Nucleosides, Penam, Penem, Phenicol, Sulfonamides, Sulfones, Tetracyclines.

Each microorganism was growth in at least two antibiotic mediums, in which the improved method predicted resistance, and at least two antibiotic mediums in which the improved method predicted susceptibility, in order to corroborate the obtained results. The results are shown in Tables 4 to 8.

TABLE 4
Antibiotic susceptibility and resistance of the microorganism
isolated in sample 1 (Klebsiella pneumoniae).

| | Prediction utilizing the | |
|---|---|---|
| Antibiotic | improved method | Result |
| Cephalosporins | Resistant | Resistant |
| (Ceftriaxone) | | |
| Fluoroquinolones | Resistant | Resistant |
| (Ciprofloxacin) | | |
| Penicillin (Methicillin) | Not resistant | Not resistant |
| Aminoglycosides (Amikacin) | Not resistant | Not resistant |

TABLE 5
Antibiotic susceptibility and resistance of the microorganism
isolated in sample 2 (Staphylococcus aureus).

| | Prediction utilizing the | |
|---|---|---|
| Antibiotic | improved method | Result |
| Macrolides | Resistant | Resistant |
| (azithromycin) | | |
| Fluoroquinolones | Resistant | Resistant |
| (Ciprofloxacin) | | |
| Penicillin (Methicillin) | Not resistant | Not resistant |
| Aminoglycosides (Amikacin) | Not resistant | Not resistant |

TABLE 6
Antibiotic susceptibility and resistance of the microorganism
isolated in sample 3 (Enterococcus faecalis).

| | Prediction utilizing the | |
|---|---|---|
| Antibiotic | improved method | Result |
| Lincosamides | Resistant | Resistant |
| (Clindamycin) | | |
| Tetracyclines | Resistant | Resistant |
| (Oxytetracycline) | | |
| Fluoroquinolones | Not resistant | Not resistant |
| (Ciprofloxacin) | | |
| Aminoglycosides (Amikacin) | Not resistant | Not resistant |

TABLE 7
Antibiotic susceptibility and resistance of the microorganism
isolated in sample 4 (Staphylococcus epidermidis).

| | Prediction utilizing the | |
|---|---|---|
| Antibiotic | improved method | Result |
| Cephalosporins | Resistant | Resistant |
| (Ceftriaxone) | | |
| Fluoroquinolones | Resistant | Resistant |
| (Ciprofloxacin) | | |
| Penicillin (Methicillin) | Not resistant | Not resistant |
| Aminoglycosides (Amikacin) | Not resistant | Not resistant |

TABLE 8
Antibiotic susceptibility and resistance of the microorganism
isolated in sample 5 (Escherichia coli).

| | Prediction utilizing the | |
|---|---|---|
| Antibiotic | improved method | Result |
| Cephalosporins | Resistant | Resistant |
| (Ceftriaxone) | | |
| Fluoroquinolones | Resistant | Resistant |
| (Ciprofloxacin) | | |
| Penicillin (Methicillin) | Not resistant | Not resistant |
| Aminoglycosides (Amikacin) | Not resistant | Not resistant |

With this example, it was demonstrated that the improved method and system using any of the devices proposed in the present invention can identify sepsis-causing microorganism, its genus and species, as well as genes that confer resistance to antibiotic. In addition, the improved method and system proposed in the present invention can perform these identifications in less than fifteen minutes.

### Example 3. Identification of Bacteria and Fungi from Soil Samples

The following example has as objective to demonstrate that the improved method and system of the present invention detects microorganisms in environment samples without doing a culture in less than ten minutes.

In order to demonstrate that the improved method and system proposed here works for any type of samples, a sample was taken from a pine forest, and after that, the DNA extraction was done using the Power Soil DNA Isolation kit (Qiagen), following supplier instructions. Nucleic Acids sequencing was done through the Miniseq Sequencing System (Illumina Inc.), following supplier instructions.

The database applied was built through ITS fungi sequences (1,236), fungi gene 18S (1,482), bacteria gene 16S (3,476), bacteria gene rpoB (2,014), gene recA (1,792), and gene gyrB (1,654). In total, database was built using 11,654 sequences

For this example, results using only a mobile phone are shown, nevertheless, results of identification obtained using any of the devices proposed in the present invention were the same.

With this database, the improved method and system proposed in the present invention could identify fifteen species of fungi and six species of bacteria in less than eight minutes from a soil sample that was sequenced from pine forest. The microorganisms that were identified are shown in the following tables 9 and 10

TABLE 9
Identified fungi from soil samples

| # | Genus | Species |
|---|---|---|
| 1 | Resinicium | bicolor |
| 2 | Penicillium | expansum |
| 3 | Lactarius | strigosipes |
| 4 | Russula | illota |
| 5 | Umbelopsis | versiformis |
| 6 | Mortierella | humillis |
| 7 | Russula | cyanoxantha |
| 8 | Fusarium | lunatum |
| 9 | Russula | aff integra |
| 10 | Inocybe | rimosa |
| 11 | Acrocalymma | vagum |
| 12 | Russula | medullata |
| 13 | Mycoleptodiscus | terrestris |
| 14 | Sistotrema | muscicola |
| 15 | Phialocephala | humicola |

TABLE 10
Identified bacteria from soil samples

| # | Genus | Species |
|---|---|---|
| 1 | Serratia | proteamaculans |
| 2 | Burkholderia | phenazinium |
| 3 | Alicyclobacillus | herbarius |
| 4 | Sphingoterrabacterium | pocheensis |
| 5 | Sphingomonas | oligophenolica |
| 6 | Bradyrhizobium | japonicum |

Taking the previous sample as an example, it is shown that the improved method and system proposed in the present invention is capable of identifying bacteria and fungi from a soil sample that has been previously sequenced

### REFERENCES

Benothman, Mohammed, Gamil A Azim, and Aboubekeur Hamdi-Cherif. 2008. «Pairwise Sequence Alignment Revisited—Genetic Algorithms and Cosine Functions». Information Technology, 9.
Buchman, Timothy G., Steven Q. Simpson, Kimberly L. Sciarretta, Kristen P. Finne, Nicole Sowers, Michael Collier, Saurabh Chavan, et al. 2020. “Sepsis Among Medicare Beneficiaries: 3. The Methods, Models, and Forecasts of Sepsis, 2012-2018*.” Critical Care Medicine 48(3):302-18.
Chatzou, Maria, Cedrik Magis, Jia-Ming Chang, Carsten Kemena, Giovanni Bussotti, Ionas Erb, and Cedric Notredame. 2016. «Multiple Sequence Alignment Modeling: Methods and Applications». Briefings in Bioinformatics 17(6):1009-23.
Fay, Katherine, Mathew R. P. Sapiano, Runa Gokhale, Raymund Dantes, Nicola Thompson, David E. Katz, Susan M. Ray, et al. 2020. “Assessment of Health Care Exposures and Outcomes in Adult Patients With Sepsis and Septic Shock.” JAMA Network Open 3(7):e206004.
Fiser, Andras. 2010. «Template-Based Protein Structure Modeling». En Computational Biology, edited by David Fenyö, 673:73-94. Methods in Molecular Biology. Totowa, NJ: Humana Press.
Needleman, Saul B., and Christian D. Wunsch. 1970. «A General Method Applicable to the Search for Similarities in the Amino Acid Sequence of Two Proteins». Journal of Molecular Biology 48(3):443-53.
Polyanovsky, Valery O, Mikhail A Roytberg, and Vladimir G Tumanyan. 2011. «Comparative Analysis of the Quality of a Global Algorithm and a Local Algorithm for Alignment of Two Sequences». Algorithms for Molecular Biology 6 (1):25.
Smith, T. F., and M. S. Waterman. 1981. «Identification of Common Molecular Subsequences». Journal of Molecular Biology 147(1): 195-97.
Yoon, Byung-Jun. 2009. «Hidden Markov Models and Their Applications in Biological Sequence Analysis». Current Genomics 10(6):402-15.

1. An improved method for identifying nucleic acid sequences present within a set of sequences obtained through a sequencer, wherein the mentioned method comprises the following steps:
Step 1).—Extraction; data obtained is extracted from a sequencer that comprises at least one or more specific sequences of DNA from a sample, wherein the obtained data are one or more nucleic acids sequences;
Step 2).—Database of specific sequences building; specific nucleic acids sequence of reference are obtained from known databases, which this specific database of sequences of reference is identified by the name of the gene:
Step 3).—Sequences loading; obtained sequences are loaded in step 1 along with database in step 2;
Step 4).—Conversion of Hash Tables; specific sequences of reference from the database that was built in step 2 are converted in one or more hash tables; wherein the specific sequence of reference of databases are converted in an exact position lists, in which each possible k-mer is located and wherein the table size has a number of elements equal to 4^{k}; the k value is a positive integer number fixed by the user, the k value is preferred between 1 and 30, more preferred between 7 and 15, and more preferred between 9 and 12;
Step 5).—Obtaining of representative k-mer from the sequences in step 1, an individual k-mer is obtained which represents each of sequence obtained in step 1, wherein the k-mer size can be similar and/or different than the selected k-mer for each Hash Table previously converted in step 4, wherein the k value is preferred between 1 and 30, more preferred between 7 and 15, and more preferred between 9 and 12;
Step 6).—Selection and exclusion, k-mer obtained in step 5 are located in Hash Tables previously converted in step 4, excluding sequences not having a position associated with a corresponding k-mer, and selecting for its later evaluation, the sequences that have one or more positions associated with the Hash Table obtained in step 4;
Step 7).—Evaluation, the sequences selected in step 6 are compared with the specific sequences of reference of the database that was built in step 2, according to the positions obtained by converting Hash Tables in step 4, wherein the comparison of each position of the two sequences must meet an evaluation criterion defined by the user, being the criterion of evaluation defined by the user equal to or greater than 90% similarity;
Step 8).—Determination of consensus sequence, sequences obtained in step 7 are analyzed in order by the position of the Hash Tables converted in step 4, applying the following expression:
S∪T=S′·A·T′
Where A is the overlapping part of the intersection of the rightmost sequences from S to T, to generate the consensus sequence;
Step 9).—Determination of the AGATA coefficient, sequences obtained in step 7 are utilized to determine AGATA coefficient, being AGATA coefficient a decimal number between 0 and 1, which 0 is the absence of similarity (0%) and 1 is the complete similarity (100%), where AGTATA coefficient is defined by the following expression:
$$CA ( α , {S}_{T} ( α ) ) = {\sum }_{k = 1}^{n} {( - 1 )}^{k + 1} \frac{\vert {⋂}_{i ∈ I ⊂ \{ 1 , … , n \} : \vert I \vert = k} {S}_{i} \vert}{\vert α \vert}$$
where α is a specific sequence of reference of the databases that were built in step 2, S_{T}(α) are the sequences selected in step 7 and C is the consensus sequence obtained in step 8 defined by
$$C = {⋃}_{k = 1}^{n} {S}_{k}$$
the above allows to obtain the AGATA coefficient for each of the specific sequences of reference of the databases that were built in step 2;
Step 10).—Identification, the AGATA coefficients obtained in step 9 allows to identify nucleic acid sequences obtained in step 1 with higher similarity to the specific sequences of reference of the databases that were built in step 2.

2. The method according to claim 1, wherein the method identifies bacteria genes, wherein the databases that were built in the step 2) comprises nucleic acid sequences from bacteria genes.

3. The method according to claim 1, wherein the method is capable of identifying yeasts genes, wherein the databases that were built in step 2) comprises nucleic acid sequences from yeasts genes

4. The method according to claim 1, wherein the method is capable of identifying virus genes, wherein the databases that were built in step 2) comprises nucleic acid sequences from virus genes.

5. The method according to claim 1, wherein the method is capable of identifying fungi genes, wherein the databases that were built in step 2) comprises nucleic acid sequences from fungi genes.

6. The method according to claim 1, wherein the method is capable of identifying plant genes, wherein the databases that were built in step 2) comprises nucleic acids from plants genes

7. The method according to claim 1, wherein the method is capable of identifying animal's genes, wherein the databases that were built in step 2) comprises nucleic acid sequences from animal's genes.

8. The method according to claim 1, wherein the method is capable of identifying human genes, wherein the databases that were built in step 2) comprises nucleic acid sequences from human genes

9. The method according to claim 1, wherein the method is capable of identifying sepsis-causing microorganism genes, wherein the databases that were built in step 2) comprises nucleic acid sequences from sepsis-causing microorganism genes.

10. The method according to claim 1, wherein the method is capable of identifying antibiotic resistance genes, wherein the databases that were built in step 2) comprises nucleic acid sequences from antibiotic resistance genes.

11. The method according to claim 1, wherein the step 2) of the method, the specific sequences of reference are obtained from known databases selected from a group that comprises pubmed, kegg, among others.

12. An improved system wherein using and analyzing metagenomic data obtained through a sequencer, identifies specific nucleic acid sequences, allowing user to modify search options to generate or modify databases capable of finding different nucleic acid sequences, characterized by the following elements:
i. The improved method according to claim 1, wherein the method is compatible with the system devices;
ii. A hard disk wherein the files related with the method and the database utilized by the system are stored;
iii. Random access memory (RAM) appropriate for loading and accessing the databases, wherein the capacity is a minimum of 2 GB, with no minimum frequency limit
iv. A processor with a minimum of 2 cores for continuous operation of the improved method

13. The improved system according to claim 12, wherein the system comprises the following elements:
I. One or more servers and/or processors units with the databases loaded, and communicated with each other through a digital network;
II. A central server and/or a central processing unit that have the database loaded, capable of communicating with computers, lap-top, tablets, iPad, iPhone, mobile phone, smartphones, or other servers or systems which could process a set of systematic operations through an installed application or app, to load, update, and delete data, to respond to informatic requests by users, implement, and/or share information and requests;
III. One or more devices selected from the group that comprises computer, lap-top, tablet, iPad, iPhone, mobile phone, smartphone, a server or servers or other system able to process a set of systematic operations through an installed application or app; and
IV. A digital informatic network that allows communication and link-up with servers, central server, or devices.

14. The improved system according to claim 12, wherein the iv) processor of the system comprises a computer-readable medium causing the processor to perform the method for identifying nucleic acid sequences,
wherein the mentioned method comprises the following steps:
Step 1).—Extraction; data obtained is extracted from a sequencer that comprises at least one or more specific sequences of DNA from a sample, wherein the obtained data are one or more nucleic acids sequences;
Step 2).—Database of specific sequences building; specific nucleic acids sequence of reference are obtained from known databases, which this specific database of sequences of reference is identified by the name of the gene:
Step 3).—Sequences loading; obtained sequences are loaded in step 1 along with database in step 2;
Step 4).—Conversion of Hash Tables; specific sequences of reference from the database that was built in step 2 are converted in one or more hash tables; wherein the specific sequence of reference of databases are converted in an exact position lists, in which each possible k-mer is located and wherein the table size has a number of elements equal to 4^{k}; the k value is a positive integer number fixed by the user, the k value is preferred between 1 and 30, more preferred between 7 and 15, and more preferred between 9 and 12;
Step 5).—Obtaining of representative k-mer from the sequences in step 1, an individual k-mer is obtained which represents each of sequence obtained in step 1, wherein the k-mer size can be similar and/or different than the selected k-mer for each Hash Table previously converted in step 4, wherein the k value is preferred between 1 and 30, more preferred between 7 and 15, and more preferred between 9 and 12;
Step 6).—Selection and exclusion, k-mer obtained in step 5 are located in Hash Tables previously converted in step 4, excluding sequences not having a position associated with a corresponding k-mer, and selecting for its later evaluation, the sequences that have one or more positions associated with the Hash Table obtained in step 4;
Step 7).—Evaluation, the sequences selected in step 6 are compared with the specific sequences of reference of the database that was built in step 2, according to the positions obtained by converting Hash Tables in step 4, wherein the comparison of each position of the two sequences must meet an evaluation criterion defined by the user, being the criterion of evaluation defined by the user equal to or greater than 90% similarity;
Step 8).—Determination of consensus sequence, sequences obtained in step 7 are analyzed in order by the position of the Hash Tables converted in step 4, applying the following expression:
S∪T=S′·A·T′
Where A is the overlapping part of the intersection of the rightmost sequences from S to T, to generate the consensus sequence;
Step 9).—Determination of the AGATA coefficient, sequences obtained in step 7 are utilized to determine AGATA coefficient, being AGATA coefficient a decimal number between 0 and 1, which 0 is the absence of similarity (0%) and 1 is the complete similarity (100%), where AGTATA coefficient is defined by the following expression:
$$CA ( α , {S}_{T} ( α ) ) = {\sum }_{k = 1}^{n} {( - 1 )}^{k + 1} \frac{\vert {⋂}_{i ∈ I ⊂ \{ 1 , … , n \} : \vert I \vert = k} {S}_{i} \vert}{\vert α \vert}$$
where α is a specific sequence of reference of the databases that were built in step 2, S_{T}(α) are the sequences selected in step 7 and C is the consensus sequence obtained in step 8 defined by
$$C = {⋃}_{k = 1}^{n} {S}_{k}$$
the above allows to obtain the AGATA coefficient for each of the specific sequences of reference of the databases that were built in step 2;
Step 10).—Identification, the AGATA coefficients obtained in step 9 allows to identify nucleic acid sequences obtained in step 1 with higher similarity to the specific sequences of reference of the databases that were built in step 2.
